(19) United States
(12) Patent Application Publication
(10) Pub. No.: US 2025/0290890 A1
(43) Pub. Date: Sep. 18, 2025

(54) DIAGNOSTICHOME KIT TODETECTMIDKINE LEVELIN BLOOD

(71) Applicant: PES University, Bengaluru (IN)

(72) Inventor: Vaishnavi Guruprasad, Bengaluru (IN)

(21) Appl. No.: 18/675,163

(22) Filed: May 28, 2024

(30) Foreign Application Priority Data
Mar. 13, 2024 (IN) 202441018279

(51) Int. Cl. G01N 27/327; G01N 33/74; G16H 50/20

(52) U.S. Cl. CPC G01N 27/3276; G01N 33/74; G16H 50/20; G01N 2333/475; G01N 2333/912

(57) ABSTRACT

The present disclosure teaches a diagnostic home kit to detect midkine level in blood samples. The kit includes an analyte receiver to receive the blood sample. Further, the kit includes a cantilever biosensor coated with a piezoelectric material and may also be immobilized by Anaplastic Lymphoma Kinase (ALK) receptors configured to attach midkine from the blood sample. The cantilevers may deflect when midkine binds to the ALK receptors, and this deflection may be captured by the piezoelectric material transducing a signal corresponding to the attached midkine. Furthermore, the kit includes an amplifier to receive and amplify the transduced signal. Moreover, the kit includes a signal processor to process the amplified signals to determine the level of midkine in the received blood sample. Additionally, the kit includes an output display unit to display midkine level in the blood sample.

### CROSS-REFERENCE TO RELATED APPLICATION

This application is based upon and claims priority to Application No. IN 202441017408, filed on 13 Mar. 2024, the entirety of which is incorporated herein by reference for all purposes.

### FIELD OF THE PRESENT DISCLOSURE

Embodiments of the present invention generally relate to the field of Biosensor Technology. In particular, embodiments of the present invention relate to the integration of biological recognition elements with sensors to detect biomarkers in biological samples.

### BACKGROUND OF THE DISCLOSURE

Midkine, a protein encoded by the MDK gene in humans, belongs to the family of heparin-binding growth factors. It actively participates in various crucial cellular processes such as cell growth, survival, and differentiation. Its significant roles extend to embryonic development, where it contributes to tissue repair and plays a part in inflammatory responses.

However, the dysregulation of midkine has been associated with a spectrum of harmful effects and the development of several diseases. Midkine has garnered attention in the realm of cardiovascular health, particularly concerning atherosclerosis and myocardial infarction. Atherosclerosis, characterized by arterial plaque buildup, has been associated with midkine's involvement in inflammation and cell proliferation, potentially influencing the progression of this vascular condition. In the context of myocardial infarction, midkine's role in tissue repair and regeneration has been explored, shedding light on its potential contributions to post-infarction recovery.

In the context of cancer, midkine's involvement becomes pronounced as it is implicated in the promotion of tumor growth, angiogenesis, and metastasis. Elevated levels of midkine are often detected in different cancer types, serving as a potential marker for disease progression. Furthermore, its overexpression correlates with unfavorable prognoses and resistance to treatment. Midkine's impact extends beyond cancer, with links to neurodegenerative diseases such as Alzheimer's and Parkinson's. In these conditions, midkine is thought to contribute to neuroinflammation and damage to neurons. The protein's pro-inflammatory properties also connect it to autoimmune conditions like rheumatoid arthritis, and inflammatory bowel disease. The cardiovascular system is not immune to midkine's influence, as it may play a role in atherosclerosis, myocardial infarction, and cardiac hypertrophy. These effects contribute to vascular inflammation and dysfunction, further complicating cardiovascular diseases. Additionally, dysregulated midkine expression is associated with renal disorders, including acute kidney injury, chronic kidney disease, and diabetic nephropathy.

Recognizing abnormalities in midkine levels emerges as a critical aspect, as it holds the potential to unveil the underlying mechanisms of these diseases. This understanding, in turn, opens avenues for the development of targeted therapeutic interventions and diagnostic strategies. In essence, midkine's intricate involvement in cellular processes underscores its significance in health and the substantial impact of its dysregulation on the pathogenesis of various diseases.

Determining midkine levels in a laboratory setting introduces several complexities in the detection process. Although established methods like ELISA and other immunoassay techniques are commonly employed, the difficulty is not uniform and can vary significantly. One significant challenge lies in the availability of specific and validated assays meticulously designed for midkine detection, as the precision of the assay directly influences the accuracy of results. The complexity is further heightened by the nuanced intricacies of the research or clinical context, where factors like the nature of the biological sample and the potential presence of interfering substances add layers of intricacy to the analysis. Additionally, the expertise of laboratory personnel plays a pivotal role, impacting the reliability of results through factors such as assay optimization, data interpretation, and troubleshooting unexpected variations. Access to specialized instrumentation is another determinant of the difficulty level, as state-of-the-art equipment enhances the precision and sensitivity of midkine detection. The overall challenge in determining midkine levels underscores the need for a comprehensive approach, combining assay specificity, contextual understanding, personnel expertise, and advanced instrumentation to ensure accurate and reliable results.

Thus, there is a need for a home kit for detecting midkine levels in the blood for diagnosing diseases, guiding treatment decisions, and understanding disease mechanisms for targeted therapies. It serves as a valuable biomarker with implications for personalized medicine and early intervention.

### BRIEF SUMMARY OF THE DISCLOSURE

An embodiment of the present disclosure teaches a diagnostic home kit to detect a level of midkine in a blood sample of a user. The user may correspond to a patient, a doctor, a guardian, a healthcare specialist, and/or a health enthusiast. In an embodiment, the diagnostic home kit includes an analyte receiver to receive the blood sample. The blood sample may be collected from the user by pricking the finger with a blood lancet. Following the puncture, a dropper may be employed to extract the blood from the finger and subsequently transfer it to the analyte receiver. Further, the home kit may, without any limitation include a biosensor, lancet, dropper, small bottle of Tris-buffered saline (TBS), and biomedical waste cover. In an embodiment, the cantilever in the biosensor includes a fixed end and a projected end. The projected end protrudes from the fixed end and is coated with a piezoelectric material. The projected end is further immobilized by a plurality of Anaplastic Lymphoma Kinase (ALK) receptors configured to attach the midkine from the received blood sample. The cantilever deflects when the midkine binds to the ALK receptors, and this deflection may be captured by the piezoelectric material transducing a signal corresponding to the attached midkine.

In an embodiment, the diagnostic home kit includes an amplifier, coupled to the fixed end of the biosensor, to receive and amplify the transduced signal. Further, the diagnostic home kit includes a signal processor to process the amplified signals to determine the level of midkine in the received blood sample. Furthermore, the diagnostic home kit includes an output display unit to display the level of midkine in the blood sample. In an embodiment, the diagnostic home kit includes an analyzer to determine a disease based on the level of midkine in the received blood sample. The determined disease is Atherosclerosis and Myocardial Infarction when the level of midkine is in a range of 50-200 ng/dL and 500-1000 ng/dL, respectively. Further, the analyzer identifies the level of midkine in a range of 0 to 0.625 ng/dL as normal. In an embodiment, the diagnostic home kit includes a network module to connect with a communication network. The network module transmits the level of midkine and the determined disease to a user device associated with a user. The user device corresponds to a mobile phone, a tablet, a computer, a laptop, a Personal Digital Assistant (PDA), a smartwatch, a smart band, and a customized healthcare device.

In an embodiment, the analyte receiver receives a buffer to wash away the attached midkine from the plurality of ALK receptors. The diagnostic home kit further includes an exit port to pull and egress out residual blood, the washed away midkine, and the buffer, wherein the buffer corresponds to a Tris-Buffered Saline (TBS).

The features and advantages of the subject matter hereof will become more apparent in light of the following detailed description of selected embodiments, as illustrated in the accompanying FIGUREs. As one of ordinary skill in the art will realize, the subject matter disclosed is capable of modifications in various respects, all without departing from the scope of the subject matter. Accordingly, the drawings and the description are to be regarded as illustrative.

### BRIEF DESCRIPTION OF THE DRAWINGS

The present subject matter will now be described in detail with reference to the drawings, which are provided as illustrative examples of the subject matter to enable those skilled in the art to practice the subject matter. It will be noted that throughout the appended drawings, features are identified by like reference numerals. Notably, the FIGUREs and examples are not meant to limit the scope of the present subject matter to a single embodiment, but other embodiments are possible by way of interchange of some or all of the described or illustrated elements and, further, wherein:

FIG. 1 illustrates an environment having a diagnostic home kit to detect midkine, according to one or more embodiments of the present disclosure.

FIG. 2 illustrates an exemplary diagnostic home kit, in accordance with an embodiment of the present disclosure.

FIG. 3 illustrates an exemplary diagnostic arrangement in the diagnostic home kit, in accordance with an embodiment of the present disclosure.

FIG. 4A-4B illustrate various interfaces of a mobile application, in accordance with an embodiment of the present disclosure.

### DETAILED DESCRIPTION OF THE EMBODIMENTS

The detailed description set forth below in connection with the appended drawings is intended as a description of exemplary embodiments in which the presently disclosed process can be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed method and system. However, it will be apparent to those skilled in the art that the presently disclosed process may be practiced without these specific details.

The terms “connected” or “coupled” and related terms are used in an operational sense and are not necessarily limited to a direct connection or coupling. Thus, for example, two devices may be coupled directly, or via one or more intermediary media or devices. As another example, devices may be coupled in such a way that information can be passed therebetween, while not sharing any physical connection. Based on the disclosure provided herein, one of ordinary skill in the art will appreciate a variety of ways in which connection or coupling exists in accordance with the aforementioned definition.

If the specification states a component or feature “may,” “can,” “could,” or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

It will be appreciated by those of ordinary skill in the art that the diagrams, schematics, illustrations, and the like represent conceptual views or processes illustrating systems and methods embodying this invention.

The present disclosure discloses a diagnostic arrangement to detect the level of midkine in blood samples which may provide rapid and easy visualization of results. The diagnostic home kit may include an analyte receiver, a cantilever based biosensor, an amplifier, a signal processor, an output display unit, and an exit port. The analyte receiver may receive the blood sample. The blood sample may be collected from the user by pricking the finger with a blood lancet. Following the puncture, a dropper may be employed to extract the blood from the finger and subsequently transfer it to the analyte receiver. The cantilever in the biosensor may include a fixed end and a projected end. The projected end may be coated with a piezoelectric material. Further, the projected end may be immobilized by a plurality of Anaplastic Lymphoma Kinase (ALK) receptors configured to attach the midkine from the received blood sample. Furthermore, the cantilever deflects when midkine binds to the ALK receptors, and this deflection may be captured by the piezoelectric material transducing a signal corresponding to the attached midkine.

In an embodiment, the amplifier may be coupled to the fixed end of the cantilever biosensor, to receive and amplify the transduced signal. The signal processor may process the amplified signals to determine the level of midkine in the received blood sample.

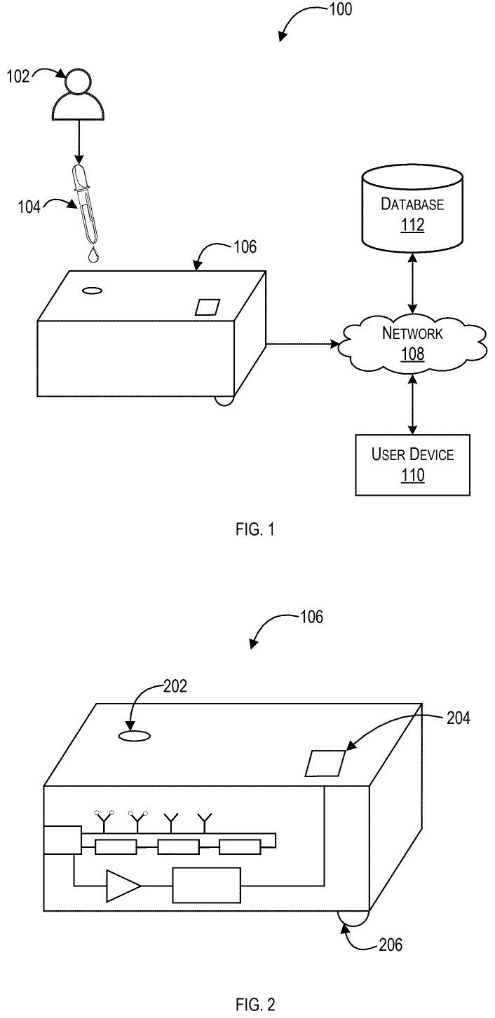

FIG. 1 illustrates an environment 100 of a diagnostic home kit to detect midkine levels in blood samples, according to one or more embodiments of the present disclosure. The environment 100 may include a user 102, a long dropper 104, the diagnostic home kit 106, a communication network 108, a database 110, and a user device 110. In an embodiment, the user 102 may correspond to a patient, a doctor, a guardian, a healthcare specialist, and/or a health enthusiast. The patient may be an individual who may assess their own symptoms or health condition without consulting a healthcare professional. Further, the user 102 may have purchased the diagnostic home kit 106 for personal use and may be interested in monitoring the midkine levels. The doctor may find value in using home kits as a convenient and rapid means of obtaining initial health data for diagnostic purposes. In certain situations, the healthcare professionals (herewith also referred to as healthcare specialists) may utilize the home kits to monitor the midkine levels in patients, assess treatment effectiveness, or integrate them into their medical practices for routine monitoring. The guardians and caregivers, responsible for individuals unable to perform tests independently, may use diagnostic home kits 106 for in-home care, and/or supervise the testing process. Additionally, healthcare specialists may employ home kits for personalized testing to determine the midkine levels, incorporating the results into treatment plans and utilizing them as part of remote monitoring protocols in virtual healthcare settings.

In an embodiment, a blood lancet may be used for the puncture or incision of the skin to obtain a small blood sample. The long dropper 104 may be utilized to obtain the blood from the finger and drop it on the analyte receiver 202. A predetermined volume of the obtained blood sample may be received by the diagnostic home kit to detect the level of midkine in the blood sample. Depending on the detected level of midkine, the user 102 may take different follow-up actions. If results are within normal ranges, the user 102 may continue with routine monitoring. If results indicate abnormalities or if there are concerns, seeking guidance from a healthcare professional may be recommended.

In an embodiment, the diagnostic home kit 106 may be coupled to a user device 110 via a communication network 108. In an embodiment, the interface of the user device 110 may include but is not limited to, a display screen, a microphone, a speaker, and so on. The user device 110 may, without any limitation, include a mobile phone, a smartphone, a Personal Assistant Device (PDA), a PC, a tablet, a laptop, a smartwatch, a smart band, a customized healthcare device, and so on. It may be understood that the transmitter may correspond to any of the existing wireless modules (e.g., Bluetooth module, Infrared module, ZigBee module, or the like) for connecting the diagnostic home kit 106 with the user device 108 via the communication network 108. Accordingly, the communication network 108 may include, without limitation, a direct interconnection, a Local Area Network (LAN), a Wide Area Network (WAN), a wireless network (e.g., using Wireless Application Protocol), the Internet, and the like.

In an embodiment, the diagnostic home kit 106 may empower user 102 to take an active role in monitoring the midkine level. Further, the diagnostic home kit 106 may provide a level of privacy that may be preferred by the user 102. The user 102 may perform the test discreetly without the need for a clinical setting. One of the key advantages for user(s) is the convenience of home testing. The users can perform the test at a time that suits them, eliminating the need to schedule appointments or visit a healthcare facility. Regular testing may prompt users to adopt healthier lifestyles or seek professional guidance when needed. It is important for users to carefully follow the instructions provided with the diagnostic home kit, understand the limitations of the test, and interpret results cautiously. If there are uncertainties or if results suggest a potential health issue, consulting with a healthcare professional ensures proper evaluation, diagnosis, and the development of an appropriate care plan.

In an embodiment, the user device 110 may communicate one or more diagnostic results to the user 102. The communicated one or more diagnostic results may include, but are not limited to, Atherosclerosis and Myocardial Infarction, etc. Further, the user device 110 may also depict qualitative and quantitative changes in the midkine level, preliminary diagnoses, and possible suggestions to improve the existing conditions.

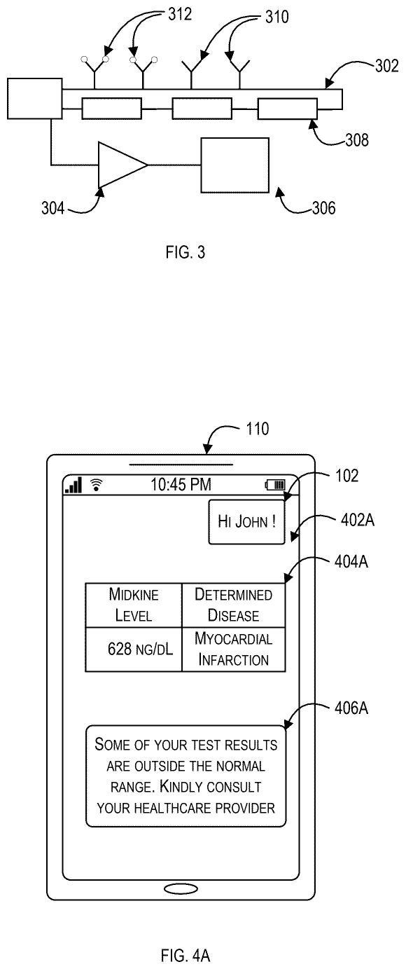

In an embodiment, a diagnostic arrangement (shown in FIG. 3) of the diagnostic home kit 106 may be exclusively arranged on a strip coated with detection markers specific to the analytes of interest. These strips when in contact with a small portion of midkine may induce the detectable changes. The information regarding the detectable changes may further be transmitted wirelessly to the user device 110, via the communication network 108.

In another embodiment, the diagnostic arrangement may detect analytes using various detection processes such as biochemical reactions, dye displacement and visual detection, nanoparticle-based sensing, and so on. If necessary, signal amplification techniques may be employed to enhance the detection process of analytes in the blood samples.

In a non-limiting embodiment, the results from the tests conducted using the diagnostic home kit may serve as a primer for medical practitioners to better advise their patients and also help patients make informed decisions. In developing economies, these tests may serve as affordable and accessible healthcare. The power of being informed and aware of one's health may help in advancing the overall health of a community.

In an embodiment, the level of midkine and/or the result to the diagnostic result may be stored in the database 112. Further, the baseline levels of midkine at normal healthy conditions may be stored in the database 112, which may be used as a reference for detection and analysis of the concentrations of midkine in the blood sample. The database 112 may also store the historical data of the user 102 for the analysis of the received information.

FIG. 2 illustrates an exemplary diagnostic home kit 106, in accordance with an embodiment of the present disclosure. FIG. 3 illustrates an exemplary diagnostic arrangement 300 in the diagnostic home kit, in accordance with an embodiment of the present disclosure. For the sake of brevity, FIG. 2 and FIG. 3 have been explained together. In an embodiment, the diagnostic home kit 106 may include an analyte receiver 202, a cantilever biosensor 302, an amplifier 304, a signal processor 306, an output display unit 204, and an exit port 206. The analyte receiver 202 may receive a predetermined volume of blood sample of the user 102. The cantilever biosensor 302 may include a fixed end and a projected end (protruding from the fixed end). The projected end may be coated with a piezoelectric material 308. The piezoelectric material 308 may be mechanically coupled to the cantilever biosensor 302. This mechanical coupling helps in efficiently transferring the mechanical stress or deformation to the piezoelectric material 308. Further, when the piezoelectric material 308 undergoes mechanical deformation due to stress, it generates an electric signal that can be measured. In an embodiment, the projected end may be immobilized by a plurality of Anaplastic Lymphoma Kinase (ALK) receptors 310 configured to attach the midkine from the received blood sample. The cantilever biosensor 302 may deflect when midkine binds to the ALK receptors, and this deflection may be captured by the piezoelectric material 308 transducing a signal corresponding to the attached midkine 312. The ALK receptors 310 may be integrated into the cantilever biosensor 302 to facilitate the detection and quantification of midkine in the received blood sample.

In an embodiment, the amplifier 304 may be coupled to the fixed end of the cantilever biosensor 302, to receive and amplify the transduced signal. Further, the amplifier 304 captures and strengthens the transduced signal generated by the piezoelectric material 308 in response to the attachment of midkine to the immobilized ALK receptors 310. This configuration is pivotal for optimizing the performance of the diagnostic home kit 106 in detecting and analyzing the level of midkine with precision and reliability. In an embodiment, the signal processor 306 may process the amplified signals to determine the level of midkine in the received blood sample. The amplified signals are employed to quantitatively measure the presence of midkine, providing valuable information about the biomarker's abundance. The signal processor 306 may translate the biosensor's response into a meaningful and measurable output, facilitating precise determination of midkine levels for diagnostic and/or research purposes.

In an embodiment, the diagnostic home kit 106 may include an analyzer (not shown in figure) to determine a disease based on the level of midkine in the received blood sample. The determined disease is Atherosclerosis and Myocardial Infarction when the level of midkine is in a range of 50-200 ng/dL and 500-1000 ng/dL, respectively. Further, the analyzer may identify the level of midkine in a range of 0 to 0.625 ng/dL as normal. In an embodiment, the output display unit 204 may display the level of midkine in the blood sample. Further, the output display unit 204 may display information or data in a visual format for users. Furthermore, the output display unit 204 may include, but not limited to, a computer monitor, screen, touchscreen interface, or any other display mechanism. Moreover, the output display unit 204 may display the information to the users in a comprehensible and accessible manner. Additionally, the output display unit 204 may present results, measurements, or other relevant data for users to interpret or act upon.

In an embodiment, the diagnostic home kit 106 may include a network module (not shown in figure) to connect with a communication network 108. Further, the network module may transmit the level of midkine and/or the determined disease to a user device 110 associated with the user 102. The inclusion of a network module in the diagnostic home kit 106 may be paramount for its seamless integration into modern healthcare ecosystems. Furthermore, the network module may enable the diagnostic kit to communicate efficiently with external devices, systems, networks, and healthcare providers. Utilizing the communication network 108 such as Wi-Fi, Bluetooth, or cellular networks, the network module ensures the secure and reliable transmission of critical data and results generated by the diagnostic home kit 106. This connectivity may facilitate remote monitoring, allowing healthcare professionals to access real-time information for timely interventions. Additionally, the network module may support over-the-air updates, ensuring that the diagnostic kit's software (if any) remains current with the latest features and security enhancements. By integrating with health information systems or electronic health records, the diagnostic home kit 106 may become an integral part of a patient's medical history.

In an embodiment, a buffer may be received by the analyte receiver 202 to wash away the attached midkine 312 from the plurality of ALK receptor 310. The exit port 206 may pull and egress out at residual blood, the washed away midkine, and the buffer. Further, the buffer may correspond to a Tris-Buffered Saline (TBS). Implementing the buffers adds a sustainable and cost-effective dimension to the detection home kit. Furthermore, the buffer may be designed for multiple applications and enhances the environmental friendliness of the home kit by reducing waste associated with single-use components. The buffer's reusability aligns with the growing emphasis on sustainable healthcare practices. Moreover, incorporating the buffer contributes to the economic viability of the kit, as users can extend its lifespan through multiple testing cycles, lowering the overall per-use cost. Additionally, the buffer's efficacy remains intact across repeated applications, maintaining the high sensitivity and specificity crucial for accurate diagnostic results. This feature not only supports environmental conservation but also underscores the commitment to resource efficiency and affordability, making the detection kit a more sustainable and accessible solution for long-term use.

In an embodiment, the wastes from the exit port 206 may be disposed of in a biomedical waste cover.

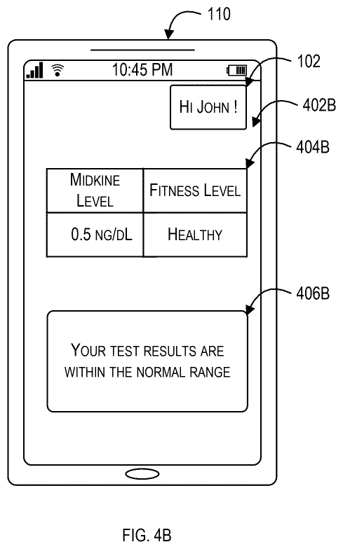

FIG. 4A-4B illustrate various interfaces of the user device 110, in accordance with an embodiment of the present disclosure. In some embodiments, the interface of the user device 110 may communicate the diagnostic result to the user 102 (such as John). The one or more diagnostic results may include, but not limited to, Atherosclerosis, Myocardial Infarction, etc. In an embodiment, when one or more test results are outside the normal limits, the interface 402A of the user device 110 may inform the user 102 of their ill health, and the information may contain some suggestions. For example, in FIG. 4A as the test results are outside or above normal limits, as shown by 404A the information ‘Some of your test results are outside the normal range’, as shown by 406A, may be communicated to the user 102. Kindly consult your healthcare provider’. In some embodiments, when the communicated results are within the normal limits, the interface 402B of the user device 110 may inform the user 102 of their well-being as shown by 404B. For example, in FIG. 4B as the test results are normal the information ‘Your test results are within the normal range’, as shown by 406B, may be communicated to the user 102.

In a non-limiting embodiment, the proposed diagnostic home kit 106 may provide results as soon as the midkine level crosses the safe limit. The diagnostic home kit 106 may be capable of measuring midkine levels representing a significant stride in the realm of healthcare with broad implications for cardiovascular health. Midkine, a multifaceted protein involved in various physiological processes, has garnered attention as a potential biomarker for cardiovascular conditions. This versatile diagnostic home kit goes beyond conventional applications by offering early detection capabilities, thereby empowering healthcare providers and individuals alike with timely information for proactive intervention. Moreover, the kit is positioned as a pivotal component in preventive care strategies, facilitating a shift towards a more pre-emptive and personalized healthcare approach. As a point-of-care solution, it provides the convenience of swift and on-the-spot results, enhancing accessibility for individuals seeking immediate insights into their cardiovascular well-being. The incorporation of midkine measurement not only contributes to risk factor assessment but also delivers comprehensive heart health insights, fostering a holistic understanding of cardiovascular conditions. In essence, this diagnostic kit is poised to reshape cardiovascular healthcare paradigms by amalgamating early detection, preventive care, and personalized insights into a single, versatile solution.

In an implementation, the diagnostic home kit, tailored to measure midkine levels, holds immense potential for a diverse range of individuals. Further, the diagnostic home kit caters to those at risk of cardiovascular diseases, offering a valuable tool for early detection and proactive management. Moreover, individuals with specific health conditions stand to benefit, as the kit's capabilities extend to providing insights relevant to their cardiovascular well-being. Furthermore, the diagnostic home kit aligns seamlessly with the goals of those focused on preventive healthcare, facilitating a comprehensive understanding of potential risks and enabling tailored strategies for wellness. Even health-conscious individuals seeking a nuanced perspective on their cardiovascular health can leverage this diagnostic solution for personalized insights and timely interventions. In essence, the incorporation of midkine measurement into this diagnostic kit transcends conventional boundaries, catering to a diverse spectrum of individuals with varying health needs and priorities.

In another implementation, the diagnostic home kit designed as a portable home kit signifies a paradigm shift, enabling users to conduct tests conveniently in the comfort of their homes. The user-friendly design enhances accessibility, ensuring that individuals, irrespective of their technical proficiency, can navigate the kit effortlessly. Rapid results emerge as a hallmark feature, providing timely information to users without unnecessary delays. Cost-effectiveness further distinguishes this kit, making advanced diagnostic capabilities more widely available. The incorporation of high sensitivity and specificity ensures accurate and reliable results, instilling confidence in the diagnostic outcomes. Notably, the kit's capability for early detection of atherosclerosis marks a groundbreaking advancement, allowing for proactive measures and interventions in cardiovascular health. In essence, this detection kit represents a holistic and innovative solution, harmonizing portability, user-friendliness, speed, cost efficiency, and precision for a transformative impact on early disease detection and management.

In an embodiment, the diagnostic home kit detects underlying chronic and systemic conditions by analyzing midkine levels. Research has indicated that elevated midkine levels may offer insights into various conditions, including cancer (lung, breast, pancreatic, neuroblastoma), neurological disorders (Alzheimer's, ischemic stroke), inflammatory diseases (rheumatoid arthritis, inflammatory bowel disease), cardiovascular diseases (atherosclerosis, myocardial infarction), and kidney diseases (diabetic nephropathy, renal fibrosis). Elevated midkine levels are linked to tumor growth, neuroinflammation, inflammation, vascular remodeling, and renal injury. By targeting midkine, the kit strives to uncover health issues that might be obscured by fluctuations in hormones, cytokines, and inflammation-related factors, thereby providing valuable diagnostic information.

Beyond midkine, the kit extends its capabilities to monitor lifestyle disorders. Through integration with a mobile application, personalized feedback and guidance are offered based on user inputs. The kit also incorporates the capacity to assess levels of lifestyle hormones and essential nutrients in the blood. In summary, this cutting-edge diagnostic home kit goes beyond midkine levels, offering a comprehensive approach to monitoring and promoting overall health. By integrating various biomarkers and adapting to individual characteristics, the kit aims to provide users with valuable information for proactive healthcare management.

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims. Thus, it will be appreciated by those of ordinary skill in the art that the diagrams, schematics, illustrations, and the like represent conceptual views or processes illustrating systems and methods embodying this disclosure.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions, or examples, which are included to enable a person having ordinary skill in the art to make and use the disclosure when combined with information and knowledge available to the person having ordinary skill in the art.

What is claimed is:

1. A diagnostic home kit to detect a level of midkine in a blood sample, the diagnostic home kit comprises:
an analyte receiver to receive the blood sample;
a cantilever biosensor having:
a fixed end; and
a projected end, protruding from the fixed end, coated with a piezoelectric material, wherein the projected end is immobilized by a plurality of Anaplastic Lymphoma Kinase (ALK) receptors configured to attach the midkine from the received blood sample, such that the piezoelectric material transduces a signal corresponding to the attached midkine;
an amplifier, coupled to the fixed end of the cantilever biosensor, to receive and amplify the transduced signal;
a signal processor to process the amplified signals to determine the level of midkine in the received blood sample.

2. The diagnostic home kit as claimed in claim 1, further comprises an output display unit to display the level of midkine in the blood sample.

3. The diagnostic home kit as claimed in claim 2, further comprises an analyzer to determine a disease based on the level of midkine in the received blood sample.

4. The diagnostic home kit as claimed in claim 3, wherein the determined disease is Atherosclerosis and Myocardial Infarction when the level of midkine is in a range of 50-200 ng/dL and 500-1000 ng/dL, respectively.

5. The diagnostic home kit as claimed in claim 3, wherein the analyzer identifies the level of midkine in a range of 0 to 0.625 ng/dL as normal.

6. The diagnostic home kit as claimed in claim 3, further comprises a network module to:
connect with a communication network; and
transmit at least one of: the level of midkine and the determined disease to a user device associated with a user.

7. The diagnostic home kit as claimed in one of claims 6, wherein the user corresponds to at least one of: a patient, a doctor, a guardian, a healthcare specialist, and a health enthusiast.

8. The diagnostic home kit as claimed in claim 6, wherein the user device corresponds to at least one of: a mobile phone, a tablet, a computer, a laptop, a Personal Digital Assistant (PDA), a smartwatch, a smart band, and a customized healthcare device.

9. The diagnostic home kit as claimed in claim 1, wherein a buffer is received by the analyte receiver to wash away the attached midkine from the plurality of ALK receptors.

10. The diagnostic home kit as claimed in claim 9, further comprises an exit port to pull and egress out at least one of: residual blood, the washed away midkine, and the buffer, wherein the buffer corresponds to a Tris-Buffered Saline (TBS).
